(19) United States
(12) Patent Application Publication
(10) Pub. No.: US 2018/0071339 A1
(43) Pub. Date: Mar. 15, 2018

(54) COMBINATION OF PHARMACEUTICAL PREPARATIONS FOR TUMOR CHEMOTHERAPY

(71) Applicant: HUBEI SOUNDNY BIOTECHNOLOGY CO., LTD., WUHAN (CN)

(72) Inventors: BO HUANG, WUHAN (CN); YI ZHANG, WUHAN (CN); JINGWEI MA, WUHAN (CN)

(21) Appl. No.: 15/785,387

(22) Filed: Oct. 16, 2017

Related U.S. Application Data
(62) Division of application No. 15/135,491, filed on Apr. 21, 2016.

(30) Foreign Application Priority Data
Apr. 21, 2015 (CN) 201510191237.7

(51) Int. Cl. A61K 35/13; A61K 45/06; A61K 47/46; A61K 31/4745; A61K 31/513; A61K 31/519; A61K 31/704; A61K 33/24

(52) U.S. Cl. CPC A61K 35/13; A61K 45/06; A61K 47/46; A61K 31/4745; A61K 2300/00; A61K 31/519; A61K 31/704; A61K 33/24; A61K 31/513

(57) ABSTRACT

The present invention provides a combination of pharmaceutical preparations for tumor chemotherapy, comprising a cell vesicles preparation with cell vesicles derived from apoptotic tumor cells for sensitizing tumor cells, and a chemotherapy drugs preparation for treating tumors. The combination of pharmaceutical preparations for tumor chemotherapy provided by the present invention uses cell vesicles derived from apoptotic tumor cells as sensitizers of tumor cells, in combination with the chemotherapy drugs preparation for treating tumors, may allow more chemotherapy drugs to enter tumor cells, so as to achieve reversal of drug resistance characteristic of tumor cells, and enhance killing effects of chemotherapy drugs against tumor cells.

### CROSS-REFERENCE TO RELATED APPLICATIONS

This application is a divisional application of U.S. patent application Ser. No. 15/135,491, filed on Apr. 21, 2016, which claims priority to Chinese Patent Application No. 201510191237.7, filed on Apr. 21, 2015. Both of the aforementioned patent applications are hereby incorporated by reference in their entireties.

### FIELD OF THE TECHNOLOGY

The present invention relates to a combination of pharmaceutical preparations for tumor chemotherapy, and particularly to a combination of pharmaceutical preparations for tumor chemotherapy comprising cell vesicles for sensitizing tumor cells and chemotherapy drugs.

### BACKGROUND

At present, using chemical drugs to destroy tumor cells is the most widely used means in clinics. However, most of the chemotherapy drugs would also cause a greater damage to normal human cells and generate side effects while killing tumor cells, so that the chemotherapy cannot be successfully carried out, thereby seriously affecting efficacy of the chemotherapy drugs. In addition, repeated using chemotherapy drugs may lead tumor cells, particularly those tumor cells with cancer stem cells characteristics to become strong drug resistance, thereby resulting in the chemotherapy drugs lose their killing effect against tumor cells. Searching for a chemotherapeutic sensitizer to enhance sensitivity of tumors towards chemotherapy drugs, such that the chemotherapy drugs have enhanced efficacy, reduced side effects, and reduced drug resistance of tumor cells, have become problems that need to be solved urgently for malignant tumors treatment.

Patients with malignant tumors usually will have multidrug resistance after experiencing repeated chemotherapy. Multidrug resistance (MDR) of tumor cells refers to tumor cells have drug resistance to one drug meanwhile generate cross-resistance to other anti-tumor drugs having different structure and different target sites. Multidrug resistance is a major cause of tumor chemotherapy failure, and it is also one of the difficult problems that need to be solved urgently in tumor chemotherapy. The mechanism for tumor MDR generation is not single, but a result of joint action of a variety of mechanisms: first of all, the generation of MDR is related to a reduction accumulation of drug in cells, while excretion of intracellular drug mainly depends on a P-gp mediated pump structure, and chemotherapy and relevant treatments can activate P-gp function, so that intracellular accumulation of drug is reduced, thereby generating tumor MDR. MDR transcriptional proteins synthesized by genes related to multidrug resistance affect distribution of drugs within cells by various mechanisms, and reduce affinity between drugs and tumor cell DNA, thereby changing the concentration gradients and pH gradients of the drugs inside and outside cells, so that drug excretion is promoted, and the tumor multidrug resistance is generated. Meanwhile, the presence of glutathione (GSH) can also protect DNA of tumor cells from attack by anti-cancer drugs through competitive inhibition in addition to the function of directly inactivation the pharmaceutical activity, thereby generating multidrug resistance. Mutation in the human topoisomerase I and II also has a direct impact on drugs and tumor cell DNA binding, generating drug resistance. And protein kinase C regulates drug distribution inside and outside the cell by five different mechanisms, to reduce drug accumulation inside the cells, thereby generating drug resistance. Apoptosis gene P53 promotes expression of MRP, thereby generating multidrug resistance of tumors, and bcl-2 prevents tumor cell necrosis by inhibiting various cytokines. CD95 reduces affinity between drug resistant cells and anti-cancer drugs and mediates the generation of tumor MDR.

How to suppress generation of multidrug resistance of tumor cells has become a hot issue in the field of anti-tumor study. Experiments found that many compounds or drugs have function of reversing MDR in vitro, but most of them have no application values in clinics trials due to their significant side effects. Accordingly, drugs, as MDR reversal agent in clinical application, have not yet been successfully developed till now. In recent years, sensitizers which may specifically improve sensitivity of tumor cells towards chemotherapy drugs, has become a new direction of seeking for a reverse MDR mechanism. People expect that these drugs themselves do not have side effects to body, but have potent killing effects on tumor cells, particularly on tumor cells with multidrug resistance, in the case of administering these drugs at or lower than a regular dosage, so as to achieve effects of controlling the development of tumors.

### SUMMARY

The present invention provides a combination of pharmaceutical preparations for tumor chemotherapy, taking a cell vesicles preparation with cell vesicles derived from apoptotic tumor cells as sensitizer, in combination with a chemotherapy drugs preparation for treating tumors, it may allow more chemotherapy drugs entering the tumors cells, thereby reversing drug resistance characteristics of tumor cells to some certain extent, and enhancing killing effects of chemotherapy drugs against tumor cells.

The invention provides a combination of pharmaceutical preparations for tumor chemotherapy, which comprises a cell vesicles preparation with cell vesicles derived from apoptotic tumor cells for sensitizing tumor cells, and a chemotherapy drugs preparation for treating tumors.

In the technical solution of the present application, taking the cell vesicles preparation and the chemotherapy drugs preparation as a whole, named as a combination of pharmaceutical preparations for tumor chemotherapy, during the process of administering the combination of pharmaceutical preparations for tumor chemotherapy of the present invention, the cell vesicles preparation is used for sensitizing tumor cells, and the chemotherapy drugs preparation is used for killing the tumor cells that have been sensitized with the cell vesicles preparation.

As basic knowledge in the field, a cell is composed of a cell membrane and cellular content wrapped therein, and the cell membrane consists of phospholipid bilayer and protein molecules embedded therein, whose spherical structure is maintained by the centripetal pulling force formed by protein fibrils within the cell, which are called cytoskeleton. When a cell is subject to stimulus of external signals (for example: chemotherapy drugs or UV light), apoptosis occurs, some protein fibrils of the cytoskeleton attached to the cell membrane site are broken, or lose adhesion, and their centripetal pulling force suddenly disappeared, such that the local cell membrane structure expands outward under the effect of the outgoing pulling force, protrudes and wraps cellular content, released to the sub-hierarchical structure between the cell and molecules in the form of cell vesicles, whose size is mostly between 100-1000 nanometers (nm), that is the “cell vesicles” (i.e. microparticles, abbreviated as Mps or Mp) as described in this invention.

In a specific embodiment of the present invention, UV light is used to induce the apoptosis of tumor cells, and as for the collection of cell vesicles, a supercentrifuge can be used for separation at low temperature or room temperature. The cell vesicles may also be collected by filtration. For example, the cell vesicles can be collected by a supercentrifuge at low temperature (4° C. or so) and at centrifugal force of 100-100,000 g. Furthermore, centrifuging cell culture medium that contains apoptotic tumor cells and collecting a first supernatant; centrifuging the first supernatant at a centrifugal force of less than 14000 g so as to remove cells and debris, and collecting a second supernatant; further centrifuging the second supernatant at a centrifugal force of 14000 g for 1 hour, collecting precipitate to obtain the cell vesicles. The cell vesicles may also be collected by filtration, for example, filtrating apoptotic tumor cells at concentration of 1×10⁹/L with a filtration membrane made of polypropylene, and pore size of the filtration membrane is 10 micrometer. After filtration, the obtained supernatant is subjected to centrifugation at centrifugal force of 14000 g for 1 hour, the precipitate is collected as cell vesicles. The collected cell vesicles may be processed into a preparation of cell vesicles, particularly injection preparation of cell vesicles according to conventional methods, for example, injection liquid, which can be produced by suspending the cell vesicles with physiologic saline.

The apoptosis of tumor cells by inducement as described in the present invention, can be judged by the criteria commonly-known by those skilled in the art, such as if the tumor cells become smaller and dimmer through observation, the cells are considered to be apoptotic cells.

The technical solutions provided in the present invention further comprise culturing the tumor cells before they are induced to apoptosis. The tumor cells comprise cells of ovarian cancer, breast cancer, lung cancer, gastric cancer, colon cancer, liver cancer, bladder cancer, or rectal cancer. The culture method for the above cells can adopt conventional culture methods in the field.

In one specific embodiment of the present invention, the chemotherapy drugs contained within the chemotherapy drugs preparation are chemotherapy drugs that contain active ingredients for treating the tumors from which the cell vesicles are provided. The chemotherapeutic drug contained also can be drugs which have already been used in clinical trials, such as injection preparation or oral preparation, if it is in the form of tablet, powder or granules and the like. For example, the chemotherapy drugs may be one or more of drugs for the treatment of ovarian cancer, breast cancer, lung cancer, gastric cancer, colon cancer, liver cancer, bladder cancer, or colorectal cancer.

Both of the cell vesicles preparation and the chemotherapy drugs preparation in the combination of pharmaceutical preparations for tumor chemotherapy of the present invention are in the form of unit preparation. Here, the unit preparation refers to a preparation that can provide active ingredient in an amount required for a single administration, such as, one unit (one needle of) injection. The amount of pharmaceutical required for a single administration to patient may be easily calculated by multiplying patient′ body weight by dose per unit of body weight of the patient. For example, during the preparation of pharmaceuticals, adult body weight is generally assumed as 50-70 kg, and using it to calculate the amount of pharmaceutical required for a single administration to patient. Dose per unit body weight of experimental animal and human may be calculated by an equivalent dose conversion relationship. For example, effective dose for human can be derived from the dose for experimental animal according to an equivalent dose conversion relationship between experimental animals and human that well-known for an ordinary skilled in the art (see, for example, guidances of FDA, SFDA and other drug regulatory authorities, or HUANG Ji-han, et al., equivalent dose conversion between different animals and between animal and human in pharmacological experiment, Chinese Journal of Clinical Pharmacology and Therapeutics, 2004 September; 9 (9): 1069-1072). In embodiments of the present invention, it can use body surface area conversion factor 0.0026 for human and mouse to derive dose for human from mouse.

Further, according to a mouse experiment by the applicant, the unit preparation of cells vesicles for human may comprise 5×10⁷-10×10⁷ cells vesicles, and the unit preparation of chemotherapy drugs for human may comprise 10-500 mg of chemotherapy drug.

Furthermore, the chemotherapy drug is doxorubicin hydrochloride, methotrexate, cisplatin, 5-fluorouracil, or hydroxycamptothecin.

Further, the dosage of the doxorubicin hydrochloride contained in the chemotherapy drugs unit preparation may be 10-50 mg.

Further, the dosage of cisplatin contained in the chemotherapy drugs unit preparation may be 20-60 mg.

Further, the dosage of methotrexate contained in the chemotherapy drugs unit preparation may be 10-50 mg.

Further, the dosage of the 5-fluorouracil contained in the chemotherapy drugs unit preparation may be 0.1-0.5 g.

Further, the amount of the hydroxycamptothecin contained in the chemotherapy drugs unit preparation may be 10-30 mg.

The combination of pharmaceutical preparations for tumor chemotherapy provided in the present invention can be administrated by firstly sensitizing the tumor cells with cell vesicles unit preparation, and then administrating the chemotherapy drugs unit preparation to the tumor cells that have been sensitized with the cell vesicles unit preparation.

The present invention also provides a method for treating tumors, including the process of administrating the combination of pharmaceutical preparations for tumor chemotherapy to patients with tumors. The invention also provides a method to prevent the generation of tumors, including the process of administrating the combination of pharmaceutical preparations for tumor chemotherapy to individuals who tend to suffer from tumors. The specific process of the method comprises firstly administering the tumor cell vesicles preparation with cell vesicles derived from apoptotic tumor cells for sensitizing, and then administrating the chemotherapy drugs preparation to the tumor cells that have been sensitized with the cell vesicles preparation, and the number ratio of cell vesicles and tumor cells is 1:3-5.

According to technical solution of the present invention, a dosage of chemotherapy drug in the combination of pharmaceutical preparations for tumor chemotherapy may be controlled according to the requirement, so as to facilitate administrating drugs to patients with different stages of tumors. The dosage of chemotherapy drug may be appropriately determined according to the nature of the selected drugs, the types of tumor that providing the cell vesicles, and tumors stages of a patient that to be treated.

In the previous studies, the applicant found that cell vesicles alone do not have killing effect to the tumor cells, only cell vesicles with chemotherapy drugs wrapped therein have killing effect to the tumor cells, and “chain reactions” also occurs: that is, after the microparticles that formed by cell vesicles wrapped the chemotherapeutic drugs therein killing the tumor cells that the cell vesicles have previously entered, the apoptotic tumor cells induced by the killing of chemotherapeutic drugs continue to form new cell vesicles wrapping the chemotherapeutic drugs, and administration of the chemotherapeutic drugs to other tumor cells continues until the medicinal effects of the chemotherapeutic drugs are fully released, such that the usage amount of chemotherapy drugs can be reduced.

However, the above studies focus on the problem of how to reduce the usage of chemotherapy drug, not how to solve the multidrug resistance of the tumor cells in the process of administering chemotherapy drugs. In addition, the above study emphasizes that administering cell vesicles and chemotherapy drugs to tumor cells at the same time, and the cell vesicles need to wrap in the chemotherapy drugs to realize the killing effect to tumor cells.

Regarding the drug resistance problem of the tumor cells in the prior art, the applicant surprisingly found that in the technical solution of the present application, when firstly administering the cell vesicles to the tumor cells in a specific amount, the cell vesicles function like sensitizers, and then administering chemotherapy drugs to tumor cells in a specific amount, which will allow more chemotherapy drugs being uptaken by the tumor cells, and the drug resistance characteristics of tumor cells can be reversed to some extent, and the killing effects of the chemotherapy drugs against tumor cells also can be improved.

The technical solutions of the present invention have the following technical effects:

1. The combination of pharmaceutical preparations for tumor chemotherapy in the present invention, which using a cell vesicles preparation with cell vesicles derived from apoptotic tumor cells in combination with a chemotherapy drugs preparation for treating tumors, which may allow more chemotherapy drugs to enter into the tumor cells, so as to reverse drug resistance characteristic of tumor cells to some extent, and enhancing the killing effects of chemotherapy drugs to tumor cells.

2. The administration of the combination of pharmaceutical preparations for tumor chemotherapy to the patients achieves the effects of effectively killing the tumor cells while reducing the toxic side-effects of the chemotherapeutic drugs against organism,

3. Tumor cells have indefinite proliferation characteristics, and their culture methods are well-developed, plurality of cell vesicles can be obtained from tumor cells according to the technical solution described in the present invention, which can be used for obtaining the combination of pharmaceutical preparations for tumor chemotherapy of the present invention, the cost is low and the operation is simple.

### BRIEF DESCRIPTION OF THE DRAWINGS

This application contains at least one drawing executed in color. Copies of this patent or patent application publication with color drawing(s) will be provided by the office upon request and payment of the necessary fee.

FIG. 1 shows a figure of cell vesicles generated from tumor cells after apoptosis treatment, observed under electronic microscope.

FIG. 2 shows effects of the combination of pharmaceutical preparations for tumor chemotherapy of the present invention on doxorubicin drugs excretion of human breast cancer cells.

FIG. 3 shows effects of the combination of pharmaceutical preparations for tumor chemotherapy of the present invention on doxorubicin drugs uptaken of MCF-7, MCF-7/TRC and ADR/MCF-7 three cells.

FIG. 4A-4B shows effects of the combination of pharmaceutical preparations for tumor chemotherapy of the present invention on expression of multidrug resistance protein P-gp.

FIG. 5 shows the combination of pharmaceutical preparations for tumor chemotherapy of the present invention has killing effects on various tumor cells.

FIG. 6 shows killing effects of the combination of pharmaceutical preparations for tumor chemotherapy of the present invention on lung cancer tumor cells in vivo.

FIG. 7A-7B shows killing effects of the combination of pharmaceutical preparations for tumor chemotherapy of the present invention on bladder cancer tumor cells in vivo.

### DETAILED DESCRIPTION

A variety of tumor cells, drugs and test animals used in the following examples:

Mouse hepatoma cells H22 (BALB/c genetic background), mouse lung cancer cell line Lewis (C57BL/6 genetic background), mouse colon cancer cell line CT-26, human breast tumor cell line MCF-7, human breast cancer adriamycin resistant strains (human breast cancer doxorubicin hydrochloride resistant strains) ADR/MCF-7, human lung cancer cell line A549, human ovarian cancer cell lines A2780, human hepatoma cell line HepG2, human gastric cancer cell lines AGS, human colorectal cancer cell line SW1116, mouse bladder cancer cell line MB49, all are commercially available from China Center for Type Culture Collection (CCTCC).

C57BL/6 mouse and BALB/c mouse are purchased from Laboratory Animal Center, College of Medicine of Wuhan University, each 5-6 weeks old, and each weighing about 16 grams.

### Example 1. Preparing the Cell Vesicles

1) Experimental Materials and Reagents

H22 mouse hepatoma cells are commercial available, UV device is available in a conventional biological safety cabinets, transmission electron microscope is JEM1010 (JEOL, Japan), and membrane brand is PALL (Part No: J100047050).

2) Experimental Procedures

H22 mouse hepatoma cancer cells are cultured in RPMI-1640 medium, so that the cell amount reaches 2×10⁷/ml; and H22 mouse hepatoma cancer cells are exposed to UV radiation for 60 minutes;

H22 mouse hepatoma cancer cells start apoptosis 18 hours after UV radiation, which is observed under microscope. Cells vesicles of the above apoptotic tumor cells are collected by centrifugation, which comprises: firstly centrifuging cell culture medium that contains apoptotic tumor cells at the rotational speed of 1300 rpm, 5000 rpm, respectively, each for 10 minutes and collecting a first supernatant; then centrifuging the first supernatant at the centrifugal force of 14000 g for 1 minute to remove cells and debris, and collecting a second supernatant; further centrifuging the second supernatant at the centrifugal force of 14000 g for 1 hour, then collecting precipitate to obtain vesicles generated from apoptotic H22 mouse hepatoma cancer cells. Alternatively, the cell culture medium that contains apoptotic tumor cells at a concentration of 1×10⁹/L is filtered, and pore size of filtration membrane is 10 microns, the supernatant obtained after filtration is centrifuged at centrifugal force of 14000 g for 1 hour, collecting precipitate to obtain the cell vesicles. Compared with centrifugation, filtration may obtain a greater number and higher purity of the cell vesicles.

3) Experimental Results

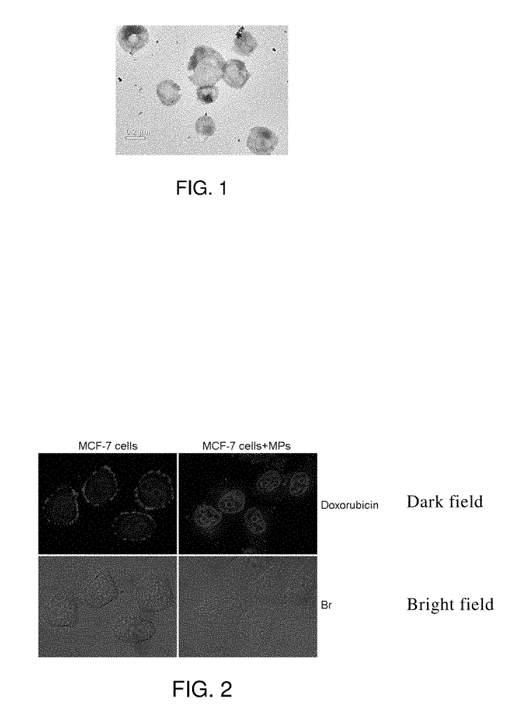

The collected cell vesicles are observed under transmission electron microscopy JEM1010 after being resuspended with 1 ml 0.9% (g/ml) of physiological saline. It can be seen from FIG. 1, the range of particle size of the cell vesicles is mostly between 100-1000 nm, cell vesicles are in the form of cavity.

### Example 2. Effects of the Combination of Pharmaceutical Preparations for Tumor Chemotherapy on Doxorubicin Drugs Excretion of Human Breast Cancer Cells

1) Experimental Materials and Reagents

Human breast cancer cell line MCF-7 and doxorubicin hydrochloride drugs are commercial available, and UV device is available in a conventional biological safety cabinets, and a Two-photon confocal microscope.

2) Experimental Procedure

Human breast cancer cell line MCF-7 is cultured in RPMI Medium 1640, so that the cell amount reaches 2×10⁷/ml; human breast cancer cell line MCF-7 is exposed to UV radiation for 60 minutes; 18 hours after UV radiation, human breast cancer cell line MCF-7 starts apoptosis, which is observed under microscope; collecting cell vesicles from the above apoptotic tumor cells by centrifugation or filtration procedure, as described in Example 1;

1×10⁵ normally cultured human breast cancer cells MCF-7 are planted in a confocal dish, and then 5×10⁵ cell vesicles prepared above and derived from human breast cancer cell line MCF-7 are added into the confocal dish and incubated with MCF-7 for 12 hours, and used as a test group (i.e., MCF-7 cells+MPs); 1×10⁵ human breast cancer cells MCF-7 without adding the cells vesicles are used as a control group (i.e., MCF-7 cells).

The test group and the control group are washed once with PBS respectively, and then a medium containing 1 μg/ml of doxorubicin drugs is added, 4 hours later, two groups are stabilized with 4% of paraformaldehyde for 20 minutes, the excretion of doxorubicin drugs from the human breast cancer cell MCF-7 in test group and control group is observed under the Two-photon confocal microscope.

3) Experimental Result

FIG. 2 shows a result of observing the excretion of doxorubicin drugs from MCF-7 human breast cancer cells in test group and control group. Control group MCF-7 human breast cancer cells that without incubating with the cell vesicles excrete a great number of drugs outside of the cell membrane, therefore drug quantity in nucleus is not high; whereas test group MCF-7 human breast cancer cells that have been incubated with cells vesicles excrete a small quantity of drugs, and large amount of doxorubicin drugs are accumulated in nucleus.

### Example 3. Effects of the Combination of Pharmaceutical Preparations for Tumor Chemotherapy on Uptake of Doxorubicin Hydrocholoride Drugs by MCF-7, MCF-7/TRC and ADR/MCF-7 Three Cells

1) Experimental Materials and Reagents

Human breast cancer cell line MCF-7, tumor repopulating cells MCF-7/TRC (tumour-repopulating cancer cell) with drug resistance, human breast cancer doxorubicin hydrochloride drug resistant strains ADR/MCF-7, three-dimensional fibrous protein gel for culturing, doxorubicin hydrochloride drugs, anti-lysosomal membrane protein lamp-2 antibody, FITC fluorescent secondary antibodies and nucleus dye DAPI, are commercially available, UV radiation device is available in a conventional biological safety cabinets, and a two-photon confocal microscopy.

2) Experimental Procedure

Cell vesicles derived from human breast cancer cell line MCF-7 are obtained in accordance with the method in Example 2;

5000 MCF-7 human breast cancer cells are planted in the three-dimensional fibrous protein gel, and after 5 days culturing, a stem cell-like colony is formed, these cells are referred to as tumor repopulating cells, that is, MCF-7/TRC. MCF-7/TRC possesses cancer stem cell characteristics, having drug resistance to a variety of chemotherapy drugs (such as cisplatin, methotrexate, 5-FU, doxorubicin hydrochloride, and the like).

1×10⁵ human breast cancer cells MCF-7, tumor repopulating cells MCF-7/TRC, human breast cancer doxorubicin hydrochloride drug resistant cells ADR/MCF-7 were planted in the confocal dish, respectively. Cell vesicles that prepared above and derived from human breast cancer cell lines MCF-7 are added to the dish containing the above three kinds of cells respectively, with amount of 5×10⁵/dish, and incubated for 12 hours, used as a test group;

Human breast cancer cells MCF-7, tumor repopulating cells MCF-7/TRC, and human breast cancer doxorubicin hydrochloride drug resistant strains ADR/MCF-7 without addition of cell vesicles are used as a control group.

MCF-7, MCF-7/TRC and ADR/MCF-7 cells in test group and control group are washed with PBS once, and then medium containing 1 μg/ml of doxorubicin hydrochloride drugs is added thereto, 4 hours later, the above groups are stabilized with 4% paraformaldehyde for 30 minutes, then immunofluorescence stained with anti-lysosomal membrane protein lamp-2 antibody, and further stained with DAPI, the uptake of doxorubicin drugs in test group MCF-7, MCF-TRC and ADR/MCF-7 cells (that is, MCF-7, MCF-7 TRCs, ADR/MCF-7) and control group MCF-7, MCF-TRC and ADR/MCF-7 cells (that is, MCF-7+MPs, TRC+MPs, ADR/MCF-7+MPs) are observed under two-photon confocal microscope.

3) Experimental Result

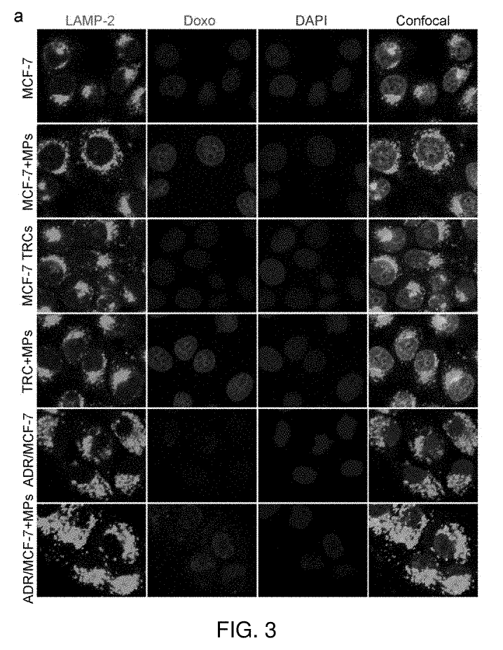

FIG. 3 shows uptake of doxorubicin drug in test group and control group MCF-7, MCF-7/TRC and ADR/MCF-7. LAMP-2 in FIG. 3 is referring to photos of cell labeled with anti-lysosomal membrane protein lamp-2 antibody, Doxo is referring to photos of cells after being administered with doxorubicin hydrocholoride, DAPI is referring to photos of cells stained with nucleus dye, confocal referring to image obtained by overlapping the above three photos. It can be seen, the doxorubicin drug content in the nucleus of control group MCF-7 cells is not high under microscope; whereas doxorubicin drug content in nucleus of test group MCF-7 cells that have been incubated with cell vesicles is significant increased. Drug content in nucleus of control group MCF-7/TRC and ADR/MCF-7 is less than that in control group MCF-7; however, drug content in nucleus of test group MCF-7/TRC and ADR/MCF-7 that have been incubated with cell vesicles is significantly increased. This result explains that being treated with cell vesicles may enhance uptake of drugs of tumor repopulating cells MCF-7/TRC with high drug resistance and doxorubicin hydrochloride drug resistant strains ADR/MCF-7.

### Example 4 Effects of Pharmaceutical Preparation for Tumor Chemotherapy on Expression of Multidrug Resistance Protein P-Gp

Experimental Materials and Reagents

Human breast cancer cell lines MCF-7, human breast cancer doxorubicin hydrochloride drug resistance strains ADR/MCF-7 and human P-gp antibody, are all commercial available, P-gp realtime PCR primers are synthesized by Shanghai Sangon Biotech Corporation.

1) Experimental Procedure

Cell vesicles derived from human breast cancer cell line MCF-7 are obtained in accordance with the method described in Example 2;

1×10⁷ cell vesicles derived from tumor cells are added to medium containing 2×10⁶ ADR/MCF-7 and incubated for 12 hours, and used as a test group; MCF-7 and ADR/MCF-7 cells without addition of cell vesicles are used as a control group.

Control group ADR/MCF-7 cells (i.e. ADR/MCF-7 cells), test group ADR/MCF-7 cells (i.e. ADR/MCF-7 cells+Mps), and control group MCF-7 cells (i.e. MCF-7 cells) are collected.

RNA extraction is performed on the above three groups, cDNA is obtained by reverse transcription of the RNA, then the expression of P-gp in the three groups of cells is detected by realtime PCR.

Sequences of the Primers are: F: SEQ ID NO:1
R: SEQ ID NO:2

PCR condition is: pre-denaturation at 95° C. for 3 minutes, and then 95° C. for 15 seconds, 60° C. for 30 seconds, conducting 40 cycles in total.

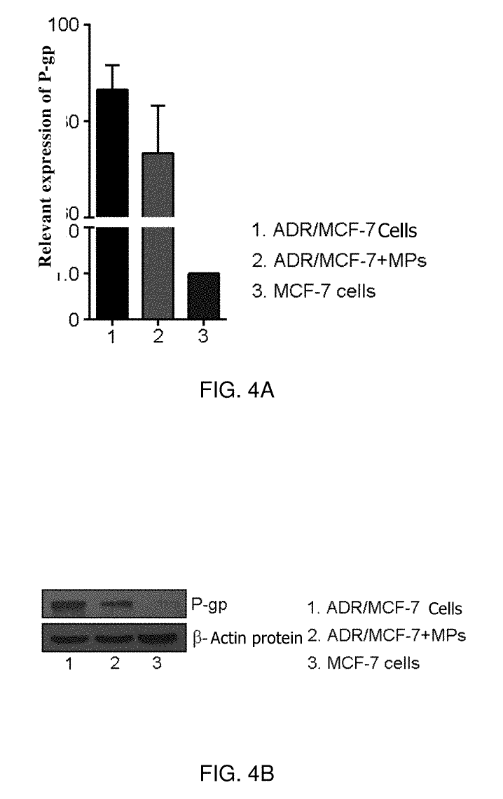

Quantitative Methods: Sybr green (fluorescent dye incorporation method), the results are shown in FIG. 4A.

Protein samples for Western blot are prepared from the three groups of cells treated by the same way.

The expression of protein P-gp in three groups of cells is detected by Western blot, the results are shown in FIG. 4B.

2) Experimental Result

As can be seen from FIG. 4A-4B, the expression level of multidrug resistance protein P-gp in test group ADR/MCR-7 cells that have been treated with cell vesicles, are all lower than that of the control group ADR/MCF-7 cells. Control group MCF-7 cells themselves have low expression of P-gp.

### Example 5 Killing Effects of Combination of Pharmaceutical Preparations for Tumor Chemotherapy of the Present Invention on Tumor Cells In Vitro

1) Experimental Materials and Reagents

Mouse hepatoma cell line H22 (BALB/c, genetic background), mouse lung cancer cell lines Lewis (C57BL/6, genetic background), mouse colon cancer cell line CT-26, human breast cancer cell line MCF-7, human lung cancer cells line A549, human ovarian cancer cell line A2780, human hepatoma cell line HepG2, human gastric cancer cell lines AGS and human colorectal cancer cell line SW1116; chemotherapy drugs are cisplatin (cisplatin, CDDP), methotrexate (MTX), 5-fluorouracil (5-Fu), and doxorubicin hydrochloride (doxorubicin, Dox), are commercially available.

2) Experimental Procedure

The above kinds of tumor cells are respectively cultured in RPMI Medium1640, and the tumor cells are cultured in a 24-well plate respectively, 5×10⁴ cells/well. Cell vesicles derived from the above kinds of tumor cells are prepared by the method in accordance with Example 1, and the cell vesicles obtained are added to same kind of tumor cells from which the cell vesicles are provided, and the number ratio of the cell vesicles and the tumor cells is 1:5, incubating for 12 hours, and used as a test group; and 5×10⁴ cells/well of the above kinds of tumor cells without addition of cell vesicles, used as a control group;

Cells in test group and control groups are washed once with PBS respectively, and then medium containing different concentration of chemotherapy drugs, 1 μg/ml of CDDP, 0.5 μg/ml of MTX, 5 μg/ml of 5-Fu, and 1 μg/ml of Dox medium, is respectively added thereto.

24 hours or 36 hours later, apoptosis of tumor cells of each group are detected by staining with AnnexinV and PI.

3) Experimental Results

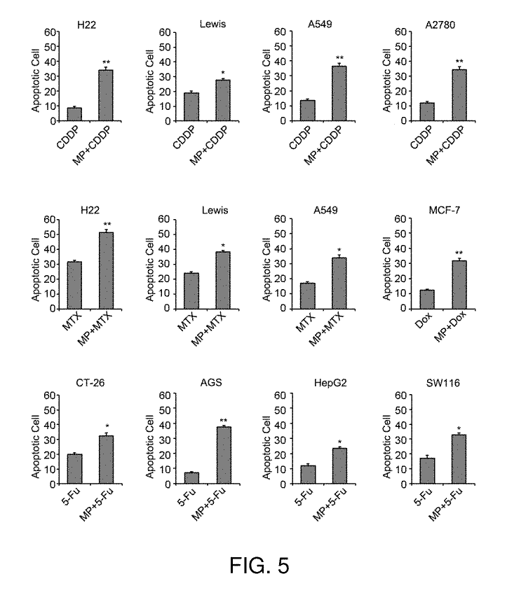

FIG. 5 shows killing effects of the combination of pharmaceutical preparations for tumor chemotherapy of the present invention on control group and test group (H22, Lewis, CT-26, MCF-7, A549, A2780, HepG2, AGS and SW1116). In comparison with the control group that using chemotherapy drugs directly killing tumor cells, the apoptosis ratio of tumor cells in test group tumor cells which have been treated with cell vesicles is higher, which indicates that the combination of pharmaceutical preparations for tumor chemotherapy of the present invention may enhance the sensitivity of tumor cells to chemotherapy drugs, thereby increasing the killing rate of tumor cells.

### Example 6 Killing Effects of the Combination of Pharmaceutical Preparations for Tumor Chemotherapy on Lung Cancer Tumor Cells

1) Experimental Materials and Reagents

C57BL/6 mouse, female, 5-6 weeks old; mouse lung cancer cell line Lewis (C57BL/6 genetic background); and methotrexate (MTX), are commercially available.

2) Experimental Procedure:

The above mouse lung cancer cells line Lewis is cultured in DMEM medium, Lewis cells are diluted into cell suspension with the PBS, and counted and diluted to 1×10⁶/300 ul, 30 C57 mice are tail vein injected with 300 ul such cell suspension, 48 hours later, grouping and administration are as follows:

Randomly dividing these mice with lung tumor into three groups, a blank group (that is control group), a drug group and a vesicle group:

The control group is injected with physiological saline every 48 h, in a total of 10 times;

The drug group (i.e. MTX) is injected with 10 μg methotrexate every 48 h, in total of 5 times;

The vesicle group (i.e. MPs+MTX) is injected with 2×10⁶ cell vesicles at 24 h, and 10 μg of methotrexate are injected at 48 h, repeat 10 times, wherein, the cell vesicles are prepared and collected in accordance with the method in Example 1, the collected cell vesicles precipitate are resuspended with 0.9% (g/ml) of physiological saline, and then prepared into injection liquid.

The mice are killed the third day after administration is finished, and their lungs are taken for observation.

3) Experimental Results

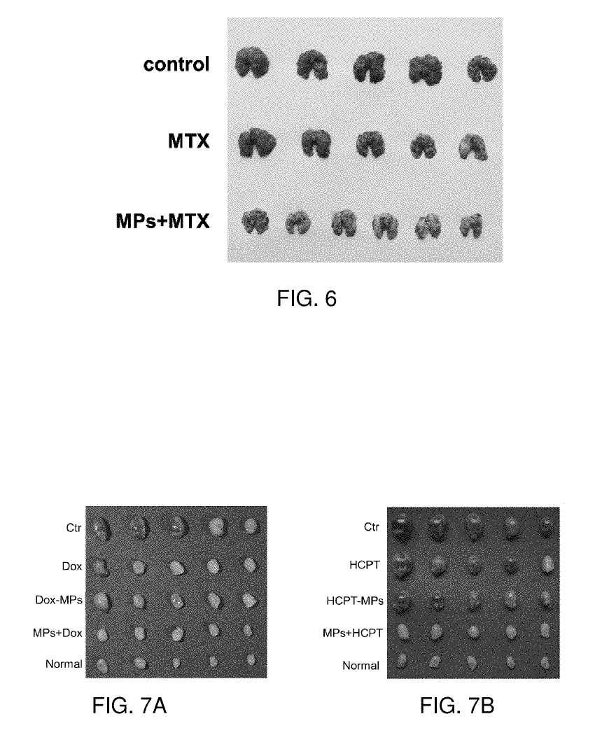

FIG. 6 shows the development of lung tumor after treated with two different combinations of preparation, MTX and MPs+MTX, wherein the lung tumor is constructed using mouse lung cancer cell Lewis. Compared with the group that treating the lung tumor with MTX alone, there are significantly improved the treatment effects in the group that firstly treating the lung tumor with MPs, then after 24 hours injected the lung tumor with MTX.

### Example 7 Killing Effects of Combination of Pharmaceutical Preparations for Tumor Chemotherapy on Bladder Tumor Cells In Vivo

1) Experimental Materials and Reagents

Mouse bladder cancer cell line MB49, polylysine, doxorubicin hydrochloride (Dox), hydroxycamptothecin (HCPT), and 6-8 weeks old female C57 mice, are commercially available.

Experimental Procedure:

Construction of Mouse Orthotopic Bladder Cancer Model:

Mouse bladder cancer cells are cultured with 10% of FBS 1640 basal medium until logarithm phase, and are resuspended with PBS into cell suspension with a cell concentration of 10⁷/mL after being digested with trypsin. After C57 mice were anesthetized, 100 uL 0.1 mg/mL of polylysin solution are infused into mouse bladder using intravenous indwelling needle for pre-treatment for 20 min, and then 100 uL cell suspension are infused into the bladder after the polylysin solution is excreted, and cell suspension is excreted after holding for 1 h.

Preparing the cell vesicles with chemotherapy drugs wrapped therein: 100 uL chemotherapy drug (Dox or HCPT) are administered to 1 ml 1×10⁷ mouse bladder cancer cells (1 mg/mL); 48 hours after administration of chemotherapy drugs, firstly centrifuging cell culture medium that contains apoptotic tumor cells at the rotational speed of 1300 rpm, 5000 rpm, respectively, each for 10 minutes and collecting a first supernatant; then centrifuging the first supernatant at the centrifugal force of 14000 g for 1 minute to remove cells and debris, and collecting a second supernatant; further centrifuging the second supernatant at the centrifugal force of 14000 g for 1 hour, then collecting precipitate to obtain cell vesicles with chemotherapy drugs wrapped therein. The cell vesicles precipitate after collection are resuspended with 0.9% (g/ml) of physiological saline, and then prepared into injection liquid.

Preparing the cell vesicles in accordance with Example 1, the collected cell vesicles precipitate are resuspended with 0.9% (g/ml) of physiological saline, and then prepared into injection liquid.

Drug Test 1:

Mice are divided into the following groups, 8 mice for each group. Control group (Ctr): it refers to mouse injected with bladder cancer cells but without treatment; Dox group: it refers to the group that administered with doxorubicin hydrochloride drug only; Dox-Mps group: it refers to MPs with doxorubicin hydrochloride drug wrapped therein; Mps+Dox group: a group firstly treated with MPs then treated with doxorubicin hydrochloride; normal group: it refers to a normal mouse group.

On day 0, mice of the above groups, except for the normal groups, are injected with tumor cells and constructed a mouse orthotopic bladder cancer model in accordance with the above method.

On day 2, 10⁶ cell vesicles are infused into every mouse with bladder cancer in Mps+Dox group, and excreted after holding it for one hour. Mice in Dox group are not infused with cell vesicles on Day 2. 10⁶ cell vesicles with doxorubicin hydrochloride drug wrapped therein are infused into bladder of mice in Dox-Mps Group on Day 2.

On the third day, 100 uL doxorubicin hydrochloride solution (1 mg/mL) is infused into bladder of mice in Dox group and Mps+Dox group, and excreted after holding it for one hour. Dox-Mps group is not subjected to the treatment.

On Day 4 and Day 5, repeat steps of Day 2 and Day 3.

On day 12, the mice are put to death, and their bladders are taken for analysis.

The control group in the whole process is only supplied with psychological saline.

Drug Test 2: Grouping and experimental processes are the same as drug test 1, except that the drug is replaced with 100 uL hydroxycamptothecin (HCPT) (1 mg/mL).

2) Experimental Results

As can be seen from FIGS. 7A and 7B, it shows no significant differences for treatment effects between mice administered with MPs having chemotherapy drug wrapped therein and mice administered with either Dox or HCPT, however, both of which has weaker treatment effects compared to mice firstly treated with cell vesicles then administered with Dox or HCPT, since treatment effects of the cell vesicles with chemotherapy drug wrapped therein depends on concentrations of chemotherapy drugs encapsulated in cell vesicles, for those drugs requires high concentration to maintain their killing effects, to enhance the killing effect, it must increase the number of cell vesicles to meet their aim, in this case, cost of treatment may increase greatly, and administering extremely high cell vesicles may also raise side effects to human body. For the purpose of reducing cost of treatment and reducing side effects on the human body caused by cell vesicles, the technical solution of present invention that firstly sensitizing the tumour cells with cell vesicles and then administering chemotherapy drugs to the sensitized tumour cells can obtain better effects, in the case of administering equal number of cell vesicles to mouse.

What is claimed is:

1. A method for treating tumors, comprising performing chemotherapy through the administration of a combination of pharmaceutical preparations for tumor chemotherapy to the patients with tumors, wherein the combination of pharmaceutical preparations for tumor chemotherapy comprising a cell vesicles preparation with cell vesicles derived from apoptotic tumor cells for sensitizing tumor cells, and a chemotherapy drugs preparation for treating tumors.

2. The method according to claim 1, wherein the chemotherapy drugs preparation comprises one or more of chemotherapy drugs for the treatment of ovarian cancer, breast cancer, lung cancer, gastric cancer, colon cancer, liver cancer, bladder cancer, or colorectal cancer.

3. The method according to claim 1, wherein particle size of the cell vesicle is 100-1000 nanometers.

4. The method according to claim 1, wherein the cell vesicles preparation and the chemotherapy drugs preparation in the combination of pharmaceutical preparations for tumor chemotherapy are in the form of unit preparation.

5. The method according to claim 4, wherein a unit preparation of cell vesicles comprises 5×10⁷-10×10⁷ of the cell vesicles, and a unit a chemotherapy drugs preparation comprises 10-500 mg of the chemotherapy drugs.

6. The method according to claim 1, wherein the cell vesicles are obtained from the following steps: using UV to irradiate the tumor cells to induce apoptosis, and collecting cell vesicles released from apoptotic tumor cells.

7. The method according to claim 6, further comprising culturing the tumor cells before they are induced to apoptosis.

8. The method according to claim 6, wherein the cell vesicles are collected by centrifugation or filtration.

9. The method according to claim 1, wherein the combination of pharmaceutical preparations for tumor chemotherapy is administrated by firstly sensitizing the tumor cells with the cell vesicles preparation, and then administrating the chemotherapy drugs preparation to the tumor cells that have been sensitized with the cell vesicles preparation.

10. The method according to claim 9, wherein the number ratio of the cell vesicles and tumor cells is 1:3-5 in the process of sensitizing.

11. The method according to claim 1, wherein the chemotherapy drugs preparation is doxorubicin hydrochloride, methotrexate, cisplatin, 5-fluorouracil, or hydroxycamptothecin.

12. The method according to claim 1, wherein the tumor cells are cells of ovarian cancer, breast cancer, lung cancer, gastric cancer, colon cancer, liver cancer, bladder cancer, or rectal cancer.


SEQUENCE LISTING (2 sequences)

SEQ ID NO: 1 - DNA, 20 bp, Artificial Sequence
  Sequence of the Primer
ccatcattgcaatagcagga

SEQ ID NO: 2 - DNA, 20 bp, Artificial Sequence
  Sequence of the Primer
ttcagtagcgatcttcccag